(19) United States
(12) Patent Application Publication
(10) Pub. No.: US 2011/0294799 A1
(43) Pub. Date: Dec. 1, 2011

(54) Organic Compounds

(75) Inventors: Samuel Hintermann, Basel (CH); Konstanze Hurth, St. Genis Pouilly (FR); Joachim Nozulak, Heitersheim (DE); Marina Tintelnot-Blomley, Maulburg (DE)

(73) Assignee: NOVARTIS AG

(21) Appl. No.: 12/673,394

(22) PCT Filed: Aug. 12, 2008
(86) PCT No.: PCT/EP2008/060603

§ 371 Date: Feb. 12, 2010

(30) Foreign Application Priority Data
Aug. 14, 2007 (EP) 07114340.8

(51) Int. Cl. A61K 31/4745; C07D 471/14; A61K 31/5377; A61K 31/4375; A61K 31/496; A61P 25/00; A61P 25/22; A61P 25/08; A61P 25/06; A61P 25/24; A61P 25/28; A61P 25/16; A61P 25/18; A61P 1/08; C07D 471/04

(52) U.S. Cl. 514/232.8; 546/82; 544/126; 544/361; 514/293; 514/253.03

(57) ABSTRACT

The invention relates to compound of the formula I

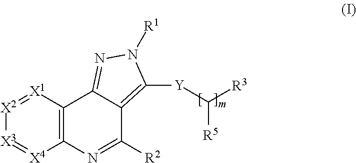

in which the substituents are as defined in the specification; in free base form or in acid addition salt form; to its preparation, to its use as medicament and to medicaments comprising it.

The present invention relates to heterocyclic compounds, to their preparation, to their use as medicaments and to medicaments comprising them.

In a first aspect, the invention relates to a compound of the formula I

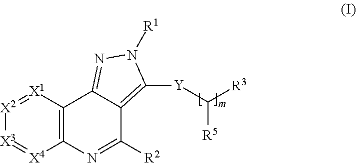

in which either
R³ represents an optionally substituted aryl group, an optionally substituted cycloalkyl group, an optionally substituted heteroaryl group, an optionally substituted heterocyclyl group; a substituted alkyl group and
m represents 0, 1, 2 or 3;
or
R³ represents hydrogen and
m represents 2, 3, 4, 5 or 6;
and
R⁵ represents hydrogen or alkyl;
Y represents O or S;
R¹ represents an optionally substituted aryl group, an optionally substituted cycloalkyl group or an optionally substituted alkyl group;
X¹ represents N, CR⁴;
X² represents N, CR⁴;
X³ represents N, CR⁴;
X⁴ represents N, CR⁴;
R⁴ represents hydrogen or a substituent different from hydrogen;
R² represents hydrogen or a substituent different from hydrogen;
and
provided that not more than two of X¹-X⁴ represent nitrogen; in free base form or in acid addition salt form.

If at least one asymmetrical carbon atom is present in a compound of the formula I, such a compound may exist in optically active form or in the form of a mixture of optical isomers, e.g. in the form of a racemic mixture. All optical isomers and their mixtures, including the racemic mixtures, are part of the present invention.

The acid addition salt of compounds of formula I are preferably pharmaceutically acceptable salts. Such salts are known in the field. As used herein, the term “pharmaceutically acceptable salts” refers to salts that retain the biological effectiveness and properties of the compounds of this invention and, which are not biologically or otherwise undesirable. In many cases, the compounds of the present invention are capable of forming acid and/or base salts by virtue of the presence of amino and/or carboxyl groups or groups similar thereto. Pharmaceutically acceptable acid addition salts can be formed with inorganic acids and organic acids, e.g., acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

The present invention includes all pharmaceutically acceptable isotopically-labeled compounds of the invention, i.e. compounds of formula (I), wherein one or more atoms are replaced by atoms having the same atomic number, but an atomic mass or mass number different from the atomic mass or mass number usually found in nature.

Examples of isotopes suitable for inclusion in the compounds of the invention comprises isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

It is further understood that, if more than one substituent R⁴ and/or R⁵ are present, each substituent may be independently selected from the list of possible substituents, i.e. one R4 may be hydrogen, other substituents R4 may be hydrogen or different from hydrogen.

The following general definitions shall apply in this specification, unless otherwise specified:

Halogen (or halo) denotes fluorine, bromine, chlorine or iodine.

Aryl is preferably naphthyl or phenyl, in particular phenyl.

Heterocyclyl represents a saturated or partly saturated ring system containing at least one hetero atom. Preferably, heterocyclyl groups consist of 3 to 11 ring atoms of which 1-3 ring atoms are hetero atoms. Heterocycles may be present as a single ring system or as bicyclic or tricyclic ring systems; preferably as single ring system or as benz-annelated ring system. Bicyclic or tricyclic ring systems may be formed by annelation of two or more rings, by a bridging atom, e.g. Oxygen, sulfur, nitrogen or by a bridging group, e.g. alkandediyl or alkenediyl. A Heterocycle may be substituted by one or more substituents selected from the group consisting of Oxo (═O), halogen, nitro, cyano, alkyl, alkandiyl, alkenediyl, alkoxy, alkoxyalkyl, alkoxycarbonyl, alkoxycarbonylalkyl, halogenalkyl, aryl, aryloxy, arylalkyl. Heteroaryl represents an aromatic ring system containing at least one hetero atom. Preferably, heteroaryl groups consist of 3 to 11 ring atoms of which 1-3 ring atoms are hetero atoms. Heteroary groups may be present as a single ring system or as bicyclic or tricyclic ring systems; preferably as single ring system or as benz-annelated ring system. Bicyclic or tricyclic ring systems may be formed by annelation of two or more rings. A Heterocycle may be substituted by one or more substituents selected from the group consisting of Oxo (═O), halogen, nitro, cyano, alkyl, alkandiyl, alkenediyl, alkoxy, alkoxyalkyl, alkoxycarbonyl, alkoxycarbonylalkyl, halogenalkyl, aryl, aryloxy, arylalkyl. Examples of heterocyclyl and heteroaryl groups include: pyrrole, pyrroline, pyrrolidine, pyrazole, pyrazoline, pyrazolidine, imidazole, imidazoline, imidazolidine, triazole, triazoline, triazolidine, tetrazole, furane, dihydrofurane, tetrahydrofurane, furazane (oxadiazole), dioxolane, thiophene, dihydrothiophene, tetrahydrothiophene, oxazole, oxazoline, oxazolidine, isoxazole, isoxazoline, isoxazolidine, thiazole, thiazoline, thiazlolidine, isothiazole, istothiazoline, isothiazolidine, thiadiazole, thiadiazoline, thiadiazolidine, pyridine, piperidine, pyridazine, pyrazine, piperazine, triazine, pyrane, tetrahydropyrane, thiopyrane, tetrahydrothiopyrane, oxazine, thiazine, dioxine, morpholine, purine, pterine, and the corresponding benz-annelated heterocycles, e.g. indole, isoindole, cumarine, cumaronecinoline, isochinoline, cinnoline.

Arylalkyl represents an aryl group bound to the molecule via an alkyl group, such as a methyl or ethyl group, preferably phenethyl or benzyl, in particular benzyl. Similarly, cycloalkylalkyl and heterocyclyl represents a cycloalkyl group bound to the molecule via an alkyl group or a heterocyclyl group bound to the molecule via an alkyl group.

Carbon containing groups, moieties or molecules contain 1 to 8, preferably 1 to 6, more preferably 1 to 4, most preferably 1 or 2, carbon atoms. Any non-cyclic carbon containing group or moiety with more than 1 carbon atom is straight-chain or branched.

Hetero atoms are atoms other than Carbon and Hydrogen, preferably nitrogen (N), oxygen (O) or sulfur (S).

Halogen-substituted groups and moieties, such as alkyl substituted by halogen (halogenalkyl) can be mono-, poly- or per-halogenated.

In preferred embodiments, which are preferred independently, collectively or in any combination or sub-combination, the invention relates to a compound of the formula I, in free base form or in acid addition salt form, wherein the substituents are as defined below.
R¹ preferably represents an optionally mono-, di-, tri- or tetra-substituted aryl group, an optionally mono-, di-, tri- or tetra-substituted cycloalkyl group or an optionally mono-, di-, tri- or tetra-substituted alkyl group; the substituent(s) being independently selected from the group consisting of halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino(C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, morpholino(C₁₋₈)alkoxy, piperidino(C₁₋₈)alkoxy, pyrrolidino(C₁₋₈)alkoxy, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl, (C₁₋₈)alkoxycarbonyl(C₁₋₈)alkoxy and —CH═CHCH═CH—, the last-mentioned optional substituent being attached to two adjacent ring carbon atoms of the said aryl group.
R¹ particular preferably represents an unsubstituted or mono-, di-, tri- or tetra-substituted aryl group, a unsubstituted or mono-, di-, tri- or tetra-substituted (C₃₋₈)cycloalkyl group or an unsubstituted, mono-, di-, tri- or tetra-substituted (C₁₋₈)alkyl group, the optional substituent(s) being independently selected from the group, consisting of halogen, (C₁₋₈)alkyl, hydroxy, (C₁₋₈)alkoxy, (C₁₋₈)alkoxy substituted by halogen, amino(C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, morpholino(C₁₋₈)alkoxy, piperidino(C₁₋₈)alkoxy, pyrrolidino(C₁₋₈)alkoxy, aminosulfonyl, (C₁₋₈)alkylaminosulfonyl, di(C₁₋₈)alkylaminosulfonyl with two identical or different (C₁₋₈)alkyl moieties, (C₁₋₈)alkoxycarbonyl(C₁₋₈).
R¹ very particular preferably represents a phenyl substituted by one or two substituents selected from the group consisting of halo, cyano, C₁-C₄ alkoxy, such as fluoro, chloro, cyano, methoxy.
R¹ further very particular preferably represents unsubstituted (C₁₋₈)alkyl or unsubstituted (C₃₋₈)cycloalkyl.
R² is preferably selected from the group consisting of hydrogen, halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino (C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl and (C₁₋₈)alkoxycarbonyl(C₁₋₈)alkoxy.
R² particular preferably represents hydrogen or (C₁₋₄)alkyl.
R² very particular preferably represents hydrogen.
R³ preferably represents an aryl group or a (C₃-C₈)cycloalkyl group or a heterocyclyl group with 3 to 8 ring atoms or a heteroaryl group with 3 to 8 ring atoms or a (C₁-C₈)alkyl group;
   wherein said aryl group, (C₃-C₈)cycloalkyl group, heteroaryl group, heterocyclyl group is unsubstituted, mono-substituted, di-substituted or tetra-substituted, the optional substituent(s) being independently selected from the group consisting of halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino(C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl, (C₁₋₈)alkoxycarbonyl(C₁₋₈)alkoxy, —OCH₂O—, —C(═O)OCH₂—, —CH₂OC(═O)— and —CH═CHCH═CH—, the four last-mentioned optional substituents in each case being attached to two adjacent ring carbon atoms of the said moiety and
      wherein said (C₁₋₈)alkyl group is mono-substituted or di-substituted, the optional substituent(s) on the said (C₁₋₈)alkyl moiety being independently selected from the group consisting of halogen, cyano, oxo, (C₁₋₈)alkoxy, (C₁₋₈)alkoxy(C₁₋₈)-alkoxy, (C₁₋₈)alkylthio, (C₁₋₈)alkylsulfinyl, (C₁₋₈) alkylsulfonyl, (C₁₋₈)alkylcarbonyloxy, (C₁₋₈)alkoxycarbonyl and (C₁₋₈)alkoxy carbonyloxy.
R³ particular preferably represents an aryl group or a (C₃-C₈)cycloalkyl group or a heteroaryl group with 5 or 6 ring atoms, or a heterocyclyl group with 5 or 6 ring atoms,
   which is unsubstituted or mono-, di-, tri- or tetra-substituted on the aryl group, the optional substituent(s) on said moiety being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy;
   which is unsubstituted or mono-, di-, tri- or tetra-substituted on the (C₃-C₈)cycloalkyl group, the optional substituent(s) on said group being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy;
   which is unsubstituted or mono-, di-, tri- or tetra-substituted on the heteroaryl group, the optional substituent(s) on the said group being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy; and whereby the heterocyclylmoiety is contains 1-3 nitrogen atoms or 0-2 nitrogen and one oxygen atom;
   which is unsubstituted or mono-, di-, tri- or tetra-substituted on the heterocyclyl group, the optional substituent(s) on the said group being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy; and whereby the heterocyclylmoiety is contains 1-3 nitrogen atoms or 0-2 nitrogen and one oxygen atom.
R³(CHR⁵)ₘ— further preferably represents ethyl, n-, iso-propyl, n-, iso-, sec.-, tert.-butyl, n-, sec.-neo.-, iso-pentyl, n-, iso-, sec.-hexyl.
Each R⁴ is independently and preferably selected from the group consisting of hydrogen, halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino, (C₁₋₈)alkoxy, (C₁₋₈)alkylamino (C₁₋₈)alkoxy, di(C₁₋₈)alkylamino (C₁₋₈)alkoxy with two identical or different (C₁₋₈)alkyl moieties, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl and (C₁₋₈)alkoxycarbonyl(C₁₋₈)alkoxy or heteroaryl.
Each R⁴ is independently and particular preferably selected from the group consisting of hydrogen, halogen, cyano, (C₁₋₈)alkyl, (C₁₋₈)alkyl substituted by halogen, (C₁₋₈)alkoxy, amino, (C₁₋₈)alkylamino and di(C₁₋₈)alkylamino with two identical or different (C₁₋₈)alkyl moieties;
Each R⁴ is further independently and particular preferably selected from the group consisting of imidazol, pyrazol, triazol, pyridine, pyrazine, pyrimidin, pyridazin, each optionally substituted by one or more substituents selected from the group consisting of halogen, cyano, (C₁₋₈)alkyl, (C₁₋₈)alkyl substituted by halogen.
Each R⁴ is independently and very particular preferably selected from the group consisting of hydrogen, fluoro, chloro, cyano, (C₁₋₄)alkyl, (C₁₋₄)alkyl substituted by fluoro.
R⁵ preferably represents hydrogen or (C₁₋₄)alkyl.
R⁵ particular preferably represents hydrogen or methyl.
Y preferably represents O.
m preferably represents 0, 1 or 2 if R³ is a substituent as defined above other than hydrogen.
m particular preferably represents 1 if R³ is a substituent as defined above other than hydrogen.

Each of X¹ to X⁴ preferably represents CR⁴.

In an advantageous embodiment, the invention relates to a compound of formula IA

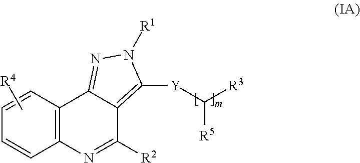

wherein the substituents are as defined for a compound of formula I.

In a further advantageous embodiment, the invention relates to a compound of formula IB

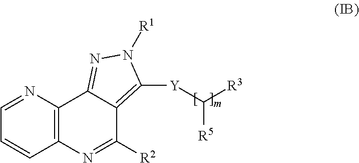

wherein the substituents are as defined for a compound of formula I.

In a further advantageous embodiment, R¹ represents a phenyl substituted in the ortho and/or para-position(s) or in the para position.

In especially preferred embodiments, the invention relates to one or more than one of the compounds of the formula I mentioned in the Examples hereinafter, in free base form or in acid addition salt form.

In a further aspect, the invention relates to a process for the preparation of the compounds of the formula I and their salts, comprising the steps of

A) reacting of a compound of the formula II

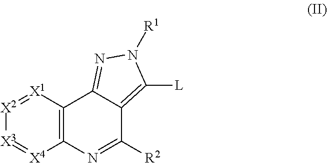

wherein the substituents are as defined for the formula I and L represents a leaving group, such as a halogen, with a compound of the formula III

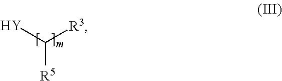

wherein the substituents are as defined for the formula I, optionally in the presence of a base, such as a hydride; optionally in the presence of one or more diluents;
or

B) reacting of a compound of the formula IV

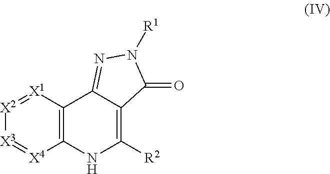

wherein the substituents are as defined for the formula I, with POCl₃
followed by a reaction with a compound of the formula III

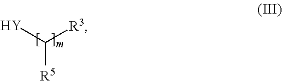

wherein the substituents are as defined for the formula I, optionally in the presence of a base, such as a hydride; optionally in the presence of one or more diluents;
and
optionally followed by reduction, oxidation or functionalization reaction of the resulting compound of formula I and/or by cleavage of protecting groups optionally present,
and
optionally followed by recovering the so obtainable compound of the formula I in free base form or in acid addition salt form.

The reactions can be effected according to conventional methods, for example as described in the Examples. The working-up of the reaction mixtures and the purification of the compounds thus obtainable may be carried out in accordance with known procedures. Acid addition salts may be produced from the free bases in known manner, and vice-versa.

Compounds of the formula I can also be prepared by further conventional processes, e.g. as described in the Examples, which processes are further aspects of the invention.

The starting materials of the formulae II, III and IV are known or may be prepared according to conventional procedures starting from known compounds, for example as described in the Examples.

Compounds of the formula I and their pharmaceutically acceptable acid addition salts, hereinafter sometimes referred to as “agents of the invention”, exhibit valuable pharmacological properties, when tested in vitro and in animals, and are, therefore, useful as active ingredients in medicaments. Agents of the invention have good efficacy as selective ligands for GABA-A receptors, showing desirable GABA-A receptor modulating activities at various receptor subtypes, and, moreover, may possess interesting pharmacokinetic properties, e.g. improved oral bioavailability or enhanced metabolic stability.

Receptors for the major inhibitory neurotransmitter, gamma aminobutyric acid (GABA), are divided into two main classes: GABA-A receptors, which are members of the ligand-gated ion channel superfamily; and GABA-B receptors, which are members of the G-protein coupled receptors superfamily. Since the first cloning of cDNAs encoding individual GABA-A receptor subunits, the number of known mammalian subunits has grown to include at least six alpha subunits, three beta subunits, three gamma subunits, three rho subunits, one delta, one epsilon, one pi, and one phi subunits. With the exception of the rho subunits which form homomultimeric receptor channels, formerly known as GABA-C receptors, it has been indicated, that a pentameric assembly of either alpha and beta subunits or alpha, beta and gamma subunits constitute the minimum requirement for forming a fully functional GABA-A receptor, when expressed by transiently transfecting cDNAs into cells. Functional receptor subtype assemblies, which do exist, include alpha1beta2gamma2, alpha2beta2gamma2 or alpha2beta3gamma2 (alpha2beta2/3gamma2), alpha3beta2/3gamma2 and alpha5beta2gamma2. Delta, epsilon, pi and phi subunits are present only to a minor extent in GABA-A receptor populations. Subtype assemblies containing an alpha1 subunit are present in most areas of the brain and are thought to account for over 40% of GABA-A receptors in the rat. Subtype assemblies containing alpha2 or alpha3 subunits, respectively, are thought to account for about 25% or 17%, respectively, of GABA-A receptors in the rat. Subtype assemblies containing alpha5 subunits are expressed predominantly in the hippocampus and the cortex. A characteristic property of all known GABA-A receptors is the presence of a number of modulatory sites. The benzodiazepine (BZD) binding site is the most explored of these, and it is the site, through which anxiolytic drugs, such as diazepam and midazolam, and hypnotic drugs, such as zolpidem and alpidem, exert their effects. It is believed, that agents acting as BZD agonists at alpha2beta2/3gamma2 and alpha3beta2/3gamma2 subtypes will possess desirable anxiolytic properties. The alpha1-selective GABA-A receptor modulators zolpidem and alpidem are clinically prescribed as hypnotic agents, suggesting that the sedation associated with known anxiolytic drugs, which act at the BZD binding site, is mediated through GABA-A receptors containing the alpha1 subunit. Compounds with inhibitory activity at the BZD site of alpha5beta2gamma2 receptor subtypes are believed to have memory improving effects.

GABA-A receptor modulators show in functional assays a positive modulation of GABA-induced signals. This modulation can be determined in vitro, e.g., at recombinant GABA-A receptors expressed in a mammalian cell line, e.g. by measurement of GABA-A receptor induced changes of the trans-membrane voltage, when using a voltage-sensitive dye and a fluorescence detection system (Adkins, C. E., Pillai, G. V., Kerby, J., Bonnert, T. P., Haldon, C., Mckernan, R. M., Gonzalez, J. E., Oades, K., Whiting, P. J. & Simpson, P. B. [2001]. alpha4beta3delta GABA-A receptors characterized by fluorescence resonance energy transfer-derived measurements of membrane potential. J. Biol. Chem., 276, 38934-38939). In this assay, a modulator compound is pre-applied at different concentrations ranging from 0.1 nM to 10 μM to cells expressing GABA-A receptors and loaded with the voltage-sensitive dye, before, or at the same time as, a sub-maximal concentration of GABA (in the range of from 0.1 to 10 μM) is applied to the cells. The fluorescent signal is correlated with the degree of GABA-A receptor channel opening. This allows the quantification of effects induced by the modulator in a functional manner. By expression of different GABA-A receptor subunit combinations, the differential efficacy of a modulator at different GABA-A receptor variants can be tested. Other functional assays include the electrophysiological recording of Xenopus oocytes or mammalian cells expressing respective receptor variants. In addition, ion flux detectors can be used to functionally study GABA-A receptors in heterologous expression systems. The affinity of a compound to the GABA-A receptor can be measured in radioligand binding experiments using reference ligands containing a radioactive element, e.g., tritiated flumazenil, and intact cells or membrane preparations of cells expressing GABA-A receptors.

Activity and selectivity of a GABA-A receptor modulator according to the invention can, e.g., be determined in vitro as follows: A transfected eukaryotic cell line expressing the alpha1, alpha2 or alpha3 subunit of the GABA-A receptor together with a beta and a gamma subunit of the GABA-A receptor is incubated with a voltage-sensitive dye, and the effects of an agonist (typically GABA) or modulator addition are recorded in a fluorimetric plate reader. The opening of the GABA-A receptor channel and the subsequent flux of anions through it changes the trans-membrane voltage of the transfected cells, leading to a change in the fluorescent signal of the voltage-sensitive dye. In the presence of the agent of the invention, a sub-maximal concentration of GABA (e.g. an EC₂₀ or an EC₅₀) added to transfected cells expressing the alpha1, alpha2 or alpha3 subunit of the GABA-A receptor will elicit an at least 50%, preferably an at least 80%, ideally an at least 100%, increase, of the fluorescent signal, compared to the fluorescent signal obtained without the agent of the invention. In this assay, agents of the invention modulate the GABA-induced response at concentration from about 0.1 to about 10′000 nM.

In vivo, a GABA-A receptor modulator can be tested in a variety of behavioral or biochemical assays, including, e.g., tests, that assess the anxiolytic-like properties, like the stress-induced hyperthermia test, the light-dark-box assay, the punished drinking (or Vogel-conflict) test, the elevated maze tests or the fear-potentiated startle response test, or tests, that assess the sedative or motor-impairing properties, like the rotarod assays, the test de traction, the primary observation test or the horizontal and vertical locomotion tests.

Due to their GABA-A receptor modulating activities, agents of the invention are useful in the treatment or prevention of a variety of disabilitating psychiatric, psychotic or neurological states, e.g. of conditions, disorders or diseases of the nervous system, that can be modulated or are mediated, fully or in part, by GABA-A receptors. Such conditions, disorders or diseases include anxiety disorders, such as panic disorder with or without agoraphobia, agoraphobia without history of panic disorder, animal or other specific phobias, including social phobias, social anxiety disorder, anxiety, obsessive-compulsive disorder, stress disorders, including post-traumatic or acute stress disorder, or generalized or substance-induced anxiety disorders; neuroses; seizures; epilepsy, especially partial seizures, simple, complex or partial seizures evolving to secondarily generalized seizures or generalized seizures [absence (typical or atypical), myoclonic, clonic, tonic, tonic-clonic or atonic seizures]; convulsions; migraine; affective disorders, including depressive or bipolar disorders, e.g. single-episode or recurrent major depressive disorder, major depression, dysthymic disorder, dysthymia, depressive disorder NOS, bipolar I or bipolar II manic disorder or cyclothymic disorder; psychotic disorders, including schizophrenia; neurodegeneration arising from cerebral ischemia; acute, traumatic or chronic degenerative processes of the nervous system, such as Parkinson's disease, Down's syndrome, senile dementia, cognitive disorders, Alzheimer's disease, Huntington's chorea, amyotrophic lateral sclerosis, multiple sclerosis or fragile X syndrome; attention disorders, e.g. attention deficit hyperactivity disorder; Tourette's syndrome; speech disorders, including stuttering; disorders of the circadian rhythm, e.g. in subjects suffering from the effects of jet lag or shift work; pain or nociception; itch; emesis, including acute, delayed or anticipatory emesis, such as emesis induced by chemotherapy or radiation, motion sickness, or post-operative nausea or vomiting; eating disorders, including anorexia nervosa or bulimia nervosa; premenstrual syndrome; muscle spasm or spasticity, e.g. in paraplegic patients; hearing disorders, e.g. tinnitus or age-related hearing impairment; urinary incontinence; or substance-related disorders, including substance abuse or dependency, including substance, such as alcohol, withdrawal disorders. Agents of the invention may also be useful in enhancing cognition, e.g. in subjects suffering from dementing conditions, such as Alzheimer's disease; as pre-medication prior to anesthesia or minor procedures, such as endoscopy, including gastric endoscopy; or as radioligands or positron emission tomography (PET) ligands in assays for detecting compounds capable of binding to the GABA-A receptor in situ.

For the above-mentioned indications, the appropriate dosage will vary depending on, e.g., the compound employed, the host, the mode of administration and the nature and severity of the condition, disorder or disease. However, in general, satisfactory results in animals are indicated to be obtained at a daily dosage of from about 0.1 to about 100, preferably from about 1 to about 50, mg/kg of animal body weight. In larger mammals, for example humans, an indicated daily dosage is in the range of from about 10 to about 2000, preferably from about 10 to about 200, mg of an agent of the invention conveniently administered, for example, in divided doses up to four times a day or in sustained release form.

An agent of the invention may be administered by any conventional route, in particular enterally, preferably orally, for example in the form of tablets or capsules, or parenterally, for example in the form of injectable solutions or suspensions.

In accordance with the foregoing, in a further aspect, the invention relates to an agent of the invention, for use as a medicament, e.g. for the treatment or prevention of conditions, disorders or diseases, that can be modulated or are mediated by GABA-A receptors.

In a further aspect, the invention relates to the use of an agent of the invention as active ingredient in a medicament, e.g. for the treatment or prevention of conditions, disorders or diseases, that can be modulated or are mediated by GABA-A receptors.

In a further aspect, the invention relates to a pharmaceutical composition comprising an agent of the invention as active ingredient in association with at least one pharmaceutical carrier or diluent. Such compositions may be manufactured in conventional manner. Unit dosage forms contain, for example, from about 1 to about 1000, preferably from about 1 to about 500, mg of an agent of the invention.

The agents of the invention can be administered alone or as combination with other pharmaceutical agents effective, e.g., in the treatment or prevention of conditions, disorders or diseases mentioned above. Such pharmaceutical combinations may be in the form of a unit dosage form, whereby each unit dosage will comprise a predetermined amount of the two components in admixture with at least one pharmaceutical carrier or diluent. Alternatively, the combination may be in the form of a package containing the two components separately, e.g. a pack or dispenser-device adapted for the concomitant or separate administration of the two active agents, wherein these agents are separately arranged. In a further aspect, the invention relates to such pharmaceutical combinations.

In a further aspect, the invention relates to the use of an agent of the invention for the manufacture of a medicament for the treatment or prevention of conditions, disorders or diseases, that can be modulated or are mediated by GABA-A receptors.

In a further aspect, the invention relates to a method for the treatment or prevention of conditions, disorders or diseases, that can be modulated or are mediated by GABA-A receptors, in a subject in need of such treatment, which comprises administering to such subject a therapeutically effective amount of an agent of the invention.

The following Examples illustrate the invention, but do not limit it.

### EXAMPLES

### Abbreviations

AcOH acetic acid
DCM dichloromethane
DMSO dimethylsulfoxide
ESIMS electrospray ionization mass spectrometry
EtOAc ethyl acetate
EtOH ethanol
h hour(s)
HPLC high pressure liquid chromatography

### LC-MS Liquid Chromatography and Mass Spectrometry

min minute(s)
NMR nuclear magnetic resonance spectrometry
rt room temperature
TFA trifluoroacetic acid
HPLC ultra performance liquid chromatography
HPLC conditions (%=percent by volume)

### Method A (Rt_{A}=Retention Time A)

UPLC Waters Acquity; column Acquity UPLC BEH C18 1.7 μm; 2.1×50 mm; gradient: 5 to 100% acetonitrile (0.1% TFA)/water (0.1% TFA), 2 min/100% acetonitrile (0.1% TFA), 0.5 min; flow 0.6 mL/min; 35° C.

### Method B (Rt_{B}=Retention Time B)

Agilent 1100 series, LC-MS; column Agilent Zorbax SB-C18; 1.8 μm; 3×30 mm; gradient: A water+0.05% TFA/B acetonitrile+0.05% TFA; from 70A:30B to 0A:100B, 3.25 min/0A:100B, 0.75 min/from 0A:100B to 70A:30B, 0.25 min; flow 0.7 mL/min; 35° C.

### Method C(Rt_{C}=Retention Time C)

Agilent 1100 series, LC-MS; column Waters Atlantis dC18; 3.5 μm; 4.6×100 mm; gradient: A water+0.05% AcOH/B acetonitrile+0.05% AcOH; from 95A:5B to 0A:100B, 8 min/0A:100B, 2 min/from 0A:100B to 95A:5B, 1 min; flow 1.5 mL/min; 35° C.

### Method D (Rt_{D}=Retention Time D)

Agilent 1100 series, LC-MS; column Waters SunFire C18; 3.5 μm; 4.6×50 mm; gradient: A water+0.05% AcOH/B acetonitrile+0.05% AcOH; from 95A:5B to 0A:100B, 4 min/0A:100B, 2 min/from 0A:100B to 95A:5B, 1 min; flow 1.5 mL/min; 35° C.

### Method E (Rt_{E}=Retention Time E)

Agilent 1100 series HPLC; column: Nucleosil C18HD (4×70 mm, 3 μm); flow: 1.0 mL/min; T=35° C.; p=50 bar; injection: 3 μl; UV-detector: 215 nm; gradient: A water+0.05% TFA/B acetonitrile+0.05% TFA; from 80A:20B to 100B in 6 min, 100B for 1.5 min, from 100B to 80A:20B in 0.5 min.

### Method F (Rt_{F}=Retention Time F)

Agilent 1100 series LC pump; Agilent 1100 series DAD; Agilent 1100 series Col Oven; CTC PAL autosampler; Waters ZQ2000 MS; column Waters XTerra C18 2.5 μm; 3×30 mm; 50° C.; mobile phase: A water 95%+acetonitrile 5%+formic acid 0.2%/B acetonitrile 100%+formic acid 0.2%; injection volume 5 μl; flow 600 μl/min; gradient 5-95% B in 3.5 min; MS parameter 100-900 Da; ESI+ cone 17V.

### Method G (Rt_{G}=Retention Time G)

Agilent 1100 series LC pump; Agilent 1100 series DAD; Agilent 1100 series Col Oven; CTC PAL autosampler; Waters ZQ2000 MS; column Waters XTerra C18 2.5 μm; 3×30 mm; 50° C.; mobile phase: A water 95%+acetonitrile 5%+formic acid 0.2%/B acetonitrile 100%+formic acid 0.2%; injection volume 5 μl; flow 600 μl/min; gradient 1-95% B in 3.5 min; MS parameter 100-900 Da; ESI+ cone 17V.

### Method H (Rt_{H}=Retention Time H)

Agilent 1100 series LC pump; Agilent 1100 series DAD; Agilent 1100 series Col Oven; CTC PAL autosampler; Waters ZQ2000 MS; column Waters XTerra C18 2.5 μm; 3×30 mm; 50° C.; mobile phase: A water 95%+acetonitrile 5%+formic acid 0.2%/B acetonitrile 100%+formic acid 0.2%; injection volume 5 μl; flow 600 μl/min; gradient 10-95% B in 1.5 min; MS parameter 100-900 Da; ESI+ cone 27V.

### Example 1

### 2-(4-Methoxy-phenyl)-3-(4-trifluoromethyl-benzyloxy)-2H-pyrazolo[4,3-c]quinoline

2-Phenylaminomethylene-malonic acid diethyl ester: 2-(Ethoxymethylene)-malonic acid diethyl ester (50 mmol) and aniline (50 mmol) are mixed in a flask. The flask is sealed, and the mixture is heated to 150° C. in a microwave reactor, stirred for 15 min and then cooled to rt. The crude oil is purified by flash-chromatography (cyclohexane/EtOAc 90:10) to yield the title compound in the form of a yellow, viscous oil.

4-Hydroxy-quinoline-3-carboxylic acid ethyl ester: 2-[(E)-Phenyliminomethyl]-malonic acid diethyl ester (48 mmol) and 60 ml of Dowtherm A are mixed in a flask. The flask is sealed, and the mixture is heated to 250° C. in a microwave reactor, stirred for 1 h, then cooled to rt and diluted with diethyl ether. The precipitating product is filtered off, washed with diethyl ether and dried at 50° C. under vacuum to yield the title compound in the form of a white solid.

4-Chloro-quinoline-3-carboxylic acid ethyl ester: 4-Hydroxy-quinoline-3-carboxylic acid ethyl ester (2.53 mmol) and 4.5 ml of POCl₃ are mixed in a flask. The flask is sealed, and the mixture is heated to 135° C., stirred for 30 min, then cooled to rt and poured onto water. The mixture is stirred for 10 min, then cooled with an ice bath, basified with aqueous NaOH solution to pH 11-12 and extracted with DCM (3 times). The combined organic fractions are dried (sodium sulfate), filtered and evaporated. The residue is purified by flash-chromatography (cyclohexane/EtOAc 60:40) to yield the title compound in the form of a light yellow solid.

2-(4-methoxy-phenyl)-1,2-dihydro-pyrazolo[4,3-c]quinolin-3-one: To 1 ml of EtOH, 4-chloroquinoline-3-carboxylic acid ethyl ester (1.00 mmol), (4-methoxyphenyl)-hydrazine (1.05 mmol) and 350 μl of triethylamine are added. The mixture is stirred in a sealed flask in a microwave reactor for 10 min at 150° C. and then for 10 min at 120° C. Then, further (4-methoxyphenyl)-hydrazine (0.5 mmol) is added. The mixture is stirred under the same conditions for 10 min at 150° C. and then slowly cooled to rt. The precipitated solid is filtered off, washed with EtOH and diethyl ether and dried under vacuum to yield the title compound in the form of a yellow solid.

2-(4-Methoxy-phenyl)-2,5-dihydro-pyrazolo[4,3-c]quinolin-3-one (20.6 mmol) is dissolved in POCl₃ (30 ml) and transferred into three closed reaction vessels. Each solution is stirred in an oil bath at 110° C. for 2.5 h and then cooled to rt. Then, all three reaction mixtures are poured onto 500 ml ice/water resulting in a yellow suspension. After 10 minutes, the pH of the solution is adjusted to 7-8 with 4N sodium hydroxide resulting in a white suspension which is filtered. The solid is washed with water and dried under vacuum to yield 3-chloro-2-(4-methoxy-phenyl)-2H-pyrazolo[4,3-c]quinoline which is used for the next step without further purification. [ESIMS [M+H]⁺=310.0; HPLC Rt_{B}=1.325 min].

2-(4-Methoxy-phenyl)-3-(4-trifluoromethyl-benzyloxy)-2H-pyrazolo[4,3-c]quinoline: In a microwave reaction vessel, 3-chloro-2-(4-methoxy-phenyl)-2H-pyrazolo[4,3-c]quinoline (0.65 mmol), 4-trifluorobenzylic alcohol (0.97 mmol, 1.5 eq.) and 0.5 M KHMDS solution (0.97 mmol, 1.5 eq.) are dissolved in THF (4 ml). The flask is sealed and the mixture heated to 100° C. in a microwave reactor. After 90 minutes, the mixture is cooled to rt and the solvent removed under vacuum. The residue is purified by flash-chromatography (0-100% EtOAc/hexane) to yield the title compound in the form of a white solid. [ESIMS [M+H]⁺=450.2; HPLC Rt_{B}=2.984 min; ¹H-NMR (CDCl₃, 600 MHz) 3.89 (s, 3H), 5.69 (s, 2H), 7.03 (d, 2H), 7.48 (d, 2H), 7.59 (t, 1H), 7.64 (d, 2H), 7.67-7.69 (m, 3H), 8.05 (d, 1H) 8.50 (d, 1H), 9.12 (s, 1H)].

### Examples 2 to 124

The compounds of Table 1 are obtainable in a manner analogous to that described in ex. 1.

TABLE 1

| | | ESIMS | |
|---|---|---|---|
| Ex. | Compound | [M + H]⁺ | HPLC |
| 1 | 2-(4-Methoxy-phenyl)-3-(4-trifluoro | 450.2 | Rt_{B} = 1.439 |
| | methyl-benzyloxy)-2H-pyrazolo[4,3-c]quinoline | | |
| 2 | 3-ethoxy-2-phenyl-2H-pyrazolo[4,3-c]quinoline | 290.2 | Rt_{B} = 1.125 |
| 3 | 2-phenyl-3-propoxy-2H-pyrazolo[4,3-c]quinoline | 304.2 | Rt_{B} = 1.22 |
| 4 | 3-butoxy-2-phenyl-2H-pyrazolo[4,3-c]quinoline | 318.2 | Rt_{B} = 1.308 |
| 5 | 2-(4-methoxyphenyl)-3-propoxy-2H-pyrazolo[4,3-c]quinoline | 334.2 | Rt_{B} = 1.215 |
| 6 | 3-ethoxy-2-(4-methoxyphenyl)-2H-pyrazolo[4,3-c]quinoline | 320.2 | Rt_{B} = 1.147 |
| 7 | 2-(2-fluorophenyl)-3-propoxy-2H-pyrazolo[4,3-c]quinoline | 322.2 | Rt_{A} = 1.19 |
| 8 | 2-(4-chlorophenyl)-4-methyl-3-(3-methylbutoxy)-2H- | 380.2 | Rt_{A} = 1.488 |
| | pyrazolo[4,3-c]quinoline | | |
| 9 | 2-(4-methoxyphenyl)-3-(3-methylbutoxy)-2H-pyrazolo[4,3- | 362.2 | Rt_{A} = 1.364 |
| | c]quinoline | | |
| 10 | 2-phenyl-3-propoxy-2H-pyrazolo[4,3-c]quinoline-6- | 329 | Rt_{H} = 1.87 |
| | carbonitrile | | |
| 11 | 2-(4-methoxyphenyl)-3-(2-methylpropoxy)-2H-pyrazolo[4,3- | 348.2 | Rt_{B} = 1.28 |
| | c]quinoline | | |
| 12 | 2-(4-methoxyphenyl)-3-(2-phenylethoxy)-2H-pyrazolo[4,3- | 396.2 | Rt_{A} = 1.356 |
| | c]quinoline | | |
| 13 | 2-(4-methoxyphenyl)-3-(2-phenoxyethoxy)-2H-pyrazolo[4,3- | 412.1 | Rt_{A} = 1.366 |
| | c]quinoline | | |
| 14 | 3-[2-(4-chlorophenyl)ethoxy]-2-(4-methoxyphenyl)-2H- | 430 | Rt_{B} = 1.449 |
| | pyrazolo[4,3-c]quinoline | | |
| 15 | 3-[2-(2-chlorophenyl)ethoxy]-2-(4-methoxyphenyl)-2H- | 430.1 | Rt_{A} = 1.412 |
| | pyrazolo[4,3-c]quinoline | | |
| 16 | 3-[2-(2-chlorophenyl)ethoxy]-2-(2-fluorophenyl)-2H- | 418.1 | Rt_{A} = 1.403 |
| | pyrazolo[4,3-c]quinoline | | |
| 17 | 1-(3-{[2-(4-methoxyphenyl)-2H-pyrazolo[4,3-c]quinolin-3- | 432.1 | Rt_{A} = 1.081 |
| | yl]oxy}propyl)-3-methylimidazolidin-2-one | | |
| 18 | 2-(4-methoxyphenyl)-3-(2-morpholin-4-ylethoxy)-2H- | 405.2 | Rt_{A} = 0.852 |
| | pyrazolo[4,3-c]quinoline | | |
| 19 | 2-(2-fluorophenyl)-3-(2-morpholin-4-ylethoxy)-2H- | 393.1 | Rt_{A} = 0.902 |
| | pyrazolo[4,3-c]quinoline | | |
| 20 | 3-(cyclohexylmethoxy)-2-(4-methoxyphenyl)-2H- | 388.2 | Rt_{A} = 1.468 |
| | pyrazolo[4,3-c]quinoline | | |
| 21 | 3-(cyclopropylmethoxy)-2-(4-methoxyphenyl)-2H- | 346.2 | Rt_{B} = 1.217 |
| | pyrazolo[4,3-c]quinoline | | |
| 22 | 3-(cyclopentylmethoxy)-2-(4-methoxyphenyl)-2H- | 374.2 | Rt_{B} = 1.413 |
| | pyrazolo[4,3-c]quinoline | | |
| 23 | 3-(cyclohexylmethoxy)-2-(2-fluorophenyl)-2H-pyrazolo[4,3- | 376.2 | Rt_{A} = 1.476 |
| | c]quinoline | | |
| 24 | 3-(cyclopentylmethoxy)-2-(2-fluorophenyl)-2H-pyrazolo[4,3- | 362.1 | Rt_{A} = 1.396 |
| | c]quinoline | | |
| 25 | 3-(cyclobutylmethoxy)-2-(4-methoxyphenyl)-2H- | 360.2 | Rt_{A} = 1.326 |
| | pyrazolo[4,3-c]quinoline | | |
| 26 | 3-(cyclobutylmethoxy)-2-(2-fluorophenyl)-2H-pyrazolo[4,3- | 348.2 | Rt_{A} = 1.32 |
| | c]quinoline | | |
| 27 | 3-[(2-fluorobenzyl)oxy]-2-phenyl-2H-pyrazolo[4,3- | 370.2 | Rt_{B} = 1.306 |
| | c]quinoline | | |
| 28 | 3-[(2-fluorobenzyl)oxy]-2-(4-fluorophenyl)-2H-pyrazolo[4,3- | 388 | Rt_{A} = 1.327 |
| | c]quinoline | | |
| 29 | 3-[(2-chlorobenzyl)oxy]-2-(2-fluorophenyl)-2H-pyrazolo[4,3- | 404.1 | Rt_{A} = 1.325 |
| | c]quinoline | | |
| 30 | 3-[(4-chlorobenzyl)oxy]-2-(2,6-dichlorophenyl)-2H- | 453.9 | Rt_{A} = 1.437 |
| | pyrazolo[4,3-c]quinoline | | |
| 31 | 3-[(4-chlorobenzyl)oxy]-6-methyl-2-phenyl-2H-pyrazolo[4,3- | 400 | Rt_{G} = 2.91 |
| | c]quinoline | | |
| 32 | 3-[(4-chlorobenzyl)oxy]-2-phenyl-2H-pyrazolo[4,3- | 411 | Rt_{G} = 3.30 |
| | c]quinoline-8-carbonitrile | | |
| 33 | 3-[(4-chlorobenzyl)oxy]-6-fluoro-2-phenyl-2H-pyrazolo[4,3- | 404 | Rt_{G} = 3.32 |
| | c]quinoline | | |
| 34 | 3-[(2-chlorobenzypoxy]-2-pyridin-2-y1-2H-pyrazolo[4,3- | 387 | Rt_{B} = 1.141 |
| | c]quinoline | | |
| 35 | 3-[(2-chlorobenzyl)oxy]-2-(2,6-dichlorophenyl)-2H- | 454 | Rt_{D} = 6.616 |
| | pyrazolo[4,3-c]quinoline | | |
| 36 | 3-[(2-bromobenzyl)oxy]-2-(2-methylphenyl)-2H- | 444 | Rt_{D} = 4.204 |
| | pyrazolo[4,3-c]quinoline | | |
| 37 | 2-(2-bromophenyl)-3-[(2-chlorobenzyl)oxy]-2H-pyrazolo[4,3- | 464 | Rt_{D} = 4.313 |
| | c]quinoline | | |
| 38 | 3-[(2-chlorobenzyl)oxy]-2-(2-ethylphenyl)-2H-pyrazolo[4,3- | 414 | Rt_{D} = 6.102 |
| | c]quinoline | | |
| 39 | 2-(2,6-dichlorophenyl)-3-[(4-fluorobenzyl)oxy]-2H- | 437.8 | Rt_{A} = 1.356 |
| | pyrazolo[4,3-c]quinoline | | |
| 40 | 3-[(2-chlorobenzyl)oxy]-2-phenyl-2H-pyrazolo[4,3- | 411 | Rt_{G} = 3.27 |
| | c]quinoline-8-carbonitrile | | |
| 41 | 3-[(2-bromobenzyl)oxy]-2-(2-chlorophenyl)-2H-pyrazolo[4,3- | 464 | Rt_{D} = 4.349 |
| | c]quinoline | | |
| 42 | 3-[(3-chlorobenzyl)oxy]-2-(2,6-dichlorophenyl)-2H- | 453.9 | Rt_{A} = 1.42 |
| | pyrazolo[4,3-c]quinoline | | |
| 43 | 2-(2,6-dichlorophenyl)-3-[(3-fluorobenzyl)oxy]-2H- | 437.9 | Rt_{A} = 1.358 |
| | pyrazolo[4,3-c]quinoline | | |
| 44 | 2-phenyl-3-{[2-(trifluoromethyl)benzyl]oxy}-2H-pyrazolo[4,3- | 420.2 | Rt_{B} = 1.393 |
| | c]quinoline | | |
| 45 | 2-(2,6-dichlorophenyl)-3-[(2-fluorobenzyl)oxy]-2H- | 437.9 | Rt_{A} = 1.33 |
| | pyrazolo[4,3-c]quinoline | | |
| 46 | 3-[(4-chlorobenzyl)oxy]-2-phenyl-2H-pyrazolo[4,3- | 411 | Rt_{H} = 1.96 |
| | c]quinoline-6-carbonitrile | | |
| 47 | 3-[(2-chlorobenzyl)oxy]-2-(2,5-dichlorophenyl)-2H- | 454 | Rt_{D} = 7.113 |
| | pyrazolo[4,3-c]quinoline | | |
| 48 | 3-[(4-chlorobenzyl)oxy]-2-(4-chlorophenyl)-9-methyl-2H- | 434 | Rt_{G} = 3.02 |
| | pyrazolo[4,3-c]quinoline | | |
| 49 | 3-[(4-tert-butylbenzyl)oxy]-2-(2,6-dichlorophenyl)-2H- | 475.9 | Rt_{A} = 1.604 |
| | pyrazolo[4,3-c]quinoline | | |
| 50 | 2-(4-fluorophenyl)-3-{[2-(trifluoromethyl)benzyl]oxy}-2H- | 437.9 | Rt_{A} = 1.415 |
| | pyrazolo[4,3-c]quinoline | | |
| 51 | 2-(2,6-dichlorophenyl)-3-{[4-(trifluoromethyl)benzyl]oxy}-2H- | 487.8 | Rt_{A} =1.483 |
| | pyrazolo[4,3-c]quinoline | | |
| 52 | 3-[(4-chlorobenzyl)oxy]-2-(4-chlorophenyl)-7- | 488 | Rt_{F} = 3.61 |
| | (trifluoromethyl)-2H-pyrazolo[4,3-c]quinoline | | |
| 53 | 3-[(2-chlorobenzyl)oxy]-2-(4-chlorophenyl)-8- | 488 | Rt_{F} = 3.61 |
| | (trifluoromethyl)-2H-pyrazolo[4,3-c]quinoline | | |
| 54 | 2-(2,6-dichlorophenyl)-3-{[3-(trifluoromethyl)benzyl]oxy}-2H- | 487.8 | Rt_{A} =1.458 |
| | pyrazolo[4,3-c]quinoline | | |
| 55 | 2-(2,6-dichlorophenyl)-3-{[2-(trifluoromethyl)benzyl]oxy}-2H- | 487.9 | Rt_{A} =1.430 |
| | pyrazolo[4,3-c]quinoline | | |
| 56 | 2-pyridin-2-yl-3-{[2-(trifluoromethyl)benzyl]oxy}-2H- | 421 | Rt_{B} = 1.188 |
| | pyrazolo[4,3-c]quinoline | | |
| 57 | 2-(4-chlorophenyl)-3-[(4-fluorobenzyl)oxy]-7- | 472 | Rt_{F} = 3.46 |
| | (trifluoromethyl)-2H-pyrazolo[4,3-c]quinoline | | |
| 58 | 2-(4-chlorophenyl)-3-[(2-fluorobenzyl)oxy]-7- | 472 | Rt_{F} = 3.49 |
| | (trifluoromethyl)-2H-pyrazolo[4,3-c]quinoline | | |
| 59 | 2-(1,3-benzodioxo1-5-y1)-3-[(2-chlorobenzypoxy]-2H- | 430 | Rt_{A} = 1.316 |
| | pyrazolo[4,3-c]quinoline | | |
| 60 | 2-(4-methoxyphenyl)-3-(pyridin-4-ylmethoxy)-2H- | 383.2 | Rt_{B} = 0.885 |
| | pyrazolo[4,3-c]quinoline | | |
| 61 | 3-[(2-chlorobenzyl)oxy]-2-(4-methoxyphenyl)-2H- | 416 | Rt_{B} = 1.343 |
| | pyrazolo[4,3-c]quinoline | | |
| 62 | 3-[(6-chloropyridin-3-yl)methoxy]-2-(4-methoxypheny1)-2H- | 417 | Rt_{B} = 1.226 |
| | pyrazolo[4,3-c]quinoline | | |
| 63 | 3-[(2-chlorobenzyl)oxy]-2-(4-fluorophenyl)-2H-pyrazolo[4,3- | 404 | Rt_{B} = 1.370 |
| | c]quinoline | | |
| 64 | 3-[(4-chlorobenzyl)oxy]-2-(2-fluorophenyl)-2H-pyrazolo[4,3- | 404.1 | Rt_{A} = 1.399 |
| | c]quinoline | | |
| 65 | 3-[(2-chlorobenzyl)oxy]-2-(4-chlorophenyl)-2H-pyrazolo[4,3- | 420 | Rt_{B} = 1.826 |
| | c]quinoline | | |
| 66 | 3-[(4-chlorobenzyl)oxy]-2-(4-chlorophenyl)-2H-pyrazolo[4,3- | 445 | Rt_{F} = 3.40 |
| | c]quinoline-7-carbonitrile | | |
| 67 | 3-[(2-chlorobenzyl)oxy]-2-(4-chlorophenyl)-2H-pyrazolo[4,3- | 445 | Rt_{F} = 3.38 |
| | c]quinoline-7-carbonitrile | | |
| 68 | 2-(4-methoxyphenyl)-3-{[3-(trifluoromethyl)benzyl]oxy}-2H- | 450.2 | Rt_{B} = 1.417 |
| | pyrazolo[4,3-c]quinoline | | |
| 69 | 3-[(4-methoxybenzyl)oxy]-2-phenyl-2H-pyrazolo[4,3- | 383 | Rt_{G} = 3.23 |
| | c][1,5]naphthyridine | | |
| 70 | 3-(benzylsulfanyl)-6,7,8-trifluoro-2-(4-methoxyphenyl)-2H- | 452 | Rt_{D} = 5.588 |
| | pyrazolo[4,3-c]quinoline | | |
| 71 | 2-(4-methoxyphenyl)-3-{[3-(trifluoromethyl)benzyl]sulfanyl} | 466 | Rt_{B} = 1.419 |
| | 2H-pyrazolo[4,3-c]quinoline | | |
| 72 | 3-(benzylsulfanyl)-2-(4-methoxyphenyl)-2H-pyrazolo[4,3- | 398.2 | Rt_{B} = 1.314 |
| | c]quinoline | | |
| 73 | 3-[(4-methoxybenzyl)sulfanyl]-2-(4-methoxyphenyl)-2H- | 428.2 | Rt_{B} = 1.309 |
| | pyrazolo[4,3-c]quinoline | | |
| 74 | 3-(benzyloxy)-2-cyclohexy1-2H-pyrazolo+4,3-c+quinoline | 358 | Rt_{c} = 4.783 |
| 75 | 3-[(2-bromobenzyl)oxy]-2-cyclohexyl-2H-pyrazolo[4,3- | 436 | Rt_{c} = 5.487 |
| | c]quinoline | | |
| 76 | 3-[(2-chlorobenzyl)oxy]-2-cyclohexyl-2H-pyrazolo[4,3- | 392 | Rt_{c} = 5.523 |
| | c]quinoline | | |
| 77 | 2-cyclohexyl-3-[(3-methylbenzyl)oxy]-2H-pyrazolo[4,3- | 372 | Rt_{c} = 5.126 |
| | c]quinoline | | |
| 78 | 3-(benzyloxy)-2-tert-butyl-2H-pyrazolo[4,3-c]quinoline | 332 | Rt_{c} = 5.451 |
| 79 | 2-tert-butyl-3-[(3-methylbenzyl)oxy]-2H-pyrazolo[4,3- | 346 | Rt_{c} = 5.777 |
| | c]quinoline | | |
| 80 | 2-tert-butyl-3-[(2-chlorobenzyl)oxy]-2H-pyrazolo[4,3- | 366 | Rt_{c} = 5.939 |
| | c]quinoline | | |
| 81 | 3-[(2-bromobenzyl)oxy]-2-tert-butyl-2H-pyrazolo[4,3- | 410 | Rt_{c} = 5.993 |
| | c]quinoline | | |
| 82 | 2-(4-Chloro-phenyl)-3-(2-fluoro-benzyloxy)-7-methyl-2H- | 419 | Rt_{F} = 2.33 |
| | pyrazolo[4,3-c][1,8]naphthyridine | | |
| 83 | 3-(2-Chloro-benzyloxy)-2-(4-chloro-phenyl)-2H- | 421 | Rt_{F} = 2.53 |
| | pyrazolo[4,3-c][1,6]naphthyridine | | |
| 84 | 3-(2-Chloro-phenoxy)-2-(4-chloro-phenyl)-2H-pyrazolo[4,3- | 406 | Rt_{F} = 2.88 |
| | c]quinoline | | |
| 85 | (3-Benzyloxy-pyrazolo[4,3-c]quinolin-2-yl)-acetic acid ethyl | 362 | Rt_{c} = 6.167 |
| | ester | | |
| 86 | [3-(2-Bromo-benzyloxy)-pyrazolo[4,3-c]quinolin-2-yl]-acetic | 440 | Rt_{c} = 6.915 |
| | acid ethyl ester | | |
| 87 | [3-(2-Chloro-benzyloxy)-pyrazolo[4,3-c]quinolin-2-yl]-acetic | 396 | Rt_{c} = 6.833 |
| | acid ethyl ester | | |
| 88 | [3-(3-Methyl-benzyloxy)-pyrazolo[4,3-c]quinolin-2-yl]-acetic | 376 | Rt_{c} = 6.688 |
| | acid ethyl ester | | |
| 89 | 3-(4-Chloro-benzyloxy)-2-(4-methoxy-phenyl)-2H- | 416 | Rt_{D} = 2.964 |
| | pyrazolo[4,3-c]quinoline | | |
| 90 | 2-(4-Chloro-phenyl)-3-cyclopropylmethoxy-2H-pyrazolo[4,3- | 350 | Rt_{A} = 1.411 |
| | c]quinoline | | |
| 91 | 2-(4-Chloro-phenyl)-3-cyclopentylmethoxy-2H-pyrazolo[4,3- | 378 | Rt_{A} = 1.620 |
| | c]quinoline | | |
| 92 | 2-(4-Chloro-phenyl)-3-cyclopentyloxy-2H-pyrazolo[4,3- | 364 | Rt_{F} = 2.34 |
| | c]quinoline | | |
| 93 | 2-(4-Chloro-phenyl)-3-(2-morpholin-4-yl-ethoxy)-2H- | 409 | Rt_{F} = 1.55 |
| | pyrazolo[4,3-c]quinoline | | |
| 94 | 2-(4-Chloro-phenyl)-3-(tetrahydro-furan-3-ylmethoxy)-2H- | 380 | Rt_{F} = 2.06 |
| | pyrazolo[4,3-c]quinoline | | |
| 95 | 2-(4-Chloro-phenyl)-3-(tetrahydro-furan-2-ylmethoxy)-2H- | 380 | Rt_{F} = 2.13 |
| | pyrazolo[4,3-c]quinoline | | |
| 96 | 2-(4-Chloro-phenyl)-3-cyclopropylmethoxy-8-imidazol-1-yl- | 416 | Rt_{E} = 3.08 |
| | 2H-pyrazolo[4,3-c]quinoline | | |
| 97 | 2-(4-Chloro-phenyl)-3-oxiranylmethoxy-2H-pyrazolo[4,3- | 352 | Rt_{F} = 2.02 |
| | c]quinoline | | |
| 98 | 2-(4-Chloro-phenyl)-3-(oxetan-2-ylmethoxy)-2H- | 366 | Rt_{F} = 2.02 |
| | pyrazolo[4,3-c]quinoline | | |
| 99 | 2-(4-Chloro-phenyl)-342-(4-methyl-piperazin-1-yl)-ethoxy]- | 422 | Rt_{F} = 1.57 |
| | 2H-pyrazolo[4,3-c]quinoline | | |
| 100 | 2-(4-Chloro-phenyl)-3-(tetrahydro-pyran-4-ylmethoxy)-2H- | 394 | Rt_{F} = 2.13 |
| | pyrazolo[4,3-c]quinoline | | |
| 101 | 3-(2-Chloro-benzyloxy)-2-(4-chloro-phenyl)-8-imidazol-1-yl- | 4.86 | Rt_{E} = 3.65 |
| | 2H-pyrazolo[4,3-c]quinoline | | |
| 102 | 2-(4-Chloro-phenyl)-3-(1-cyclopentyl-ethoxy)-2H- | 392 | Rt_{F} = 2.63 |
| | pyrazolo[4,3-c]quinoline | | |
| 103 | 2-(4-Chloro-phenyl)-3-(1-methyl-piperidin-4-yloxy)-2H- | 393 | Rt_{F} = 1.53 |
| | pyrazolo[4,3-c]quinoline | | |
| 104 | 2-(4-Chloro-phenyl)-3-(tetrahydro-pyran-4-yloxy)-2H- | 380 | Rt_{F} = 2.12 |
| | pyrazolo[4,3-c]quinoline | | |
| 105 | 2-(4-Chloro-phenyl)-3-(2-cyclohexyl-ethoxy)-2H- | 406 | Rt_{F} = 2.80 |
| | pyrazolo[4,3-c]quinoline | | |
| 106 | 2-(4-Chloro-phenyl)-3-[2-(2-fluoro-phenyl)-ethoxy]-2H- | 418 | Rt_{F} = 2.47 |
| | pyrazolo[4,3-c]quinoline | | |
| 107 | 2-(4-Chloro-phenyl)-3-(2-cyclopropyl-ethoxy)-2H- | 364 | Rt_{F} = 2.40 |
| | pyrazolo[4,3-c]quinoline | | |
| 108 | 2-(4-Chloro-phenyl)-3-[2-(2-chloro-phenyl)-ethoxy]-2H- | 434 | Rt_{F} = 2.58 |
| | pyrazolo[4,3-c]quinoline | | |
| 109 | 2-(4-Chloro-phenyl)-3-[2-(4-chloro-phenyl)-ethoxy]-2H- | 434 | Rt_{F} = 2.60 |
| | pyrazolo[4,3-c]quinoline | | |
| 110 | 2-(4-Chloro-phenyl)-3-[2-(4-fluoro-phenyl)-ethoxy]-2H- | 418 | Rt_{F} = 2.47 |
| | pyrazolo[4,3-c]quinoline | | |
| 111 | 2-(4-Chloro-phenyl)-3-phenethyloxy-2H-pyrazolo[4,3- | 400 | Rt_{F} = 2.46 |
| | c]quinoline | | |
| 112 | 2-(4-Chloro-phenyl)-3-(2-o-tolyl-ethoxy)-2H-pyrazolo[4,3- | 414 | Rt_{F} = 2.54 |
| | c]quinoline | | |
| 113 | 2-(4-Chloro-phenyl)-3-cyclopentylmethoxy-8-imidazol-1-yl- | 444 | Rt_{E} = 3.74 |
| | 2H-pyrazolo[4,3-c]quinoline | | |
| 114 | 2-(4-Chloro-phenyl)-3-[2-(2-chloro-phenyl)-ethoxy]-8- | 500 | Rt_{E} = 3.77 |
| | imidazol-1-yl-2H-pyrazolo[4,3-c]quinoline | | |
| 115 | 8-Bromo-3-(2-chloro-benzyloxy)-2-(4-chloro-phenyl)-2H- | 498 | Rt_{E} = 5.64 |
| | pyrazolo[4,3-c]quinoline | | |
| 116 | 8-Bromo-2-(4-chloro-phenyl)-3-cyclopropylmethoxy-2H- | 428 | Rt_{E} = 5.06 |
| | pyrazolo[4,3-c]quinoline | | |
| 117 | 2-(4-Chloro-phenyl)-3-cyclopropylmethoxy-8-pyrazol-1-yl- | 416 | Rt_{E} = 4.51 |
| | 2H-pyrazolo[4,3-c]quinoline | | |
| 118 | 2-(4-Chloro-phenyl)-3-(2-piperidin-1-yl-ethoxy)-2H- | 407 | Rt_{F} = 1.64 |
| | pyrazolo[4,3-c]quinoline | | |
| 119 | 2-(4-Chloro-phenyl)-3-(1-cyclohexyl-ethoxy)-2H- | 406 | Rt_{F} = 2.75 |
| | pyrazolo[4,3-c]quinoline | | |
| 120 | 2-(4-Chloro-phenyl)-3-(1-cyclohexyl-propoxy)-2H- | 420 | Rt_{F} = 2.90 |
| | pyrazolo[4,3-c]quinoline | | |
| 121 | 3-(2-Chloro-benzyloxy)-2-(4-trifluoromethoxy-phenyl)-2H- | 470 | Rt_{A} = 1.64 |
| | pyrazolo[4,3-c]quinoline | | |
| 122 | 2-(4-Chloro-phenyl)-3-(2-p-tolyl-ethoxy)-2H-pyrazolo[4,3- | 414 | Rt_{F} = 2.54 |
| | c]quinoline | | |
| 123 | 2-(4-Chloro-phenyl)-3-(2-cyclopentyl-ethoxy)-2H- | 392 | Rt_{F} = 2.64 |
| | pyrazolo[4,3-c]quinoline | | |
| 124 | 2-(4-Chloro-phenyl)-3-(1-methyl-piperidin-4-ylmethoxy)-2H- | 407 | Rt_{A} = 1.56 |
| | pyrazolo[4,3-c]quinoline | | |
| 125 | 2-(4-Chloro-phenyl)-3-(2-pyrrolidin-1-yl-ethoxy)-2H- | 393 | Rt_{A} = 1.50 |
| | pyrazolo[4,3-c]quinoline | | |

TABLE 2
The activity of agents of the invention as GABA-A
alpha2 and/or alphal receptor modulators is tested
as described above (fluorescence measurements of
transfected eukaryotic cell lines expressing the
alpha 1 or 2 subunit together with a beta and a
gamma subunit). The compounds are tested at 3 μM
and at a sub-maximal concentration of GABA (EC₂₀).
The values are expressed as “% mod” meaning a
percentage of increase of the fluorescent signal
compared to the fluorescent signal obtained without
the agent of the invention.

| | alpha1 | alpha2 |
|---|---|---|
| Ex. | % mod | % mod |
| 1 | 5 | 36 |
| 4 | 137 | 126 |
| 13 | −29 | 52 |
| 14 | 3 | 34 |
| 15 | 23 | 197 |
| 16 | 18 | 101 |
| 17 | 262 | 577 |
| 18 | 67 | 196 |
| 19 | 0 | 72 |
| 27 | 62 | 165 |
| 28 | 34 | 146 |
| 31 | 51 | 81 |
| 32 | 34 | 88 |
| 33 | 39 | 73 |
| 34 | 13 | 57 |
| 36 | 223 | 315 |
| 41 | 289 | 386 |
| 50 | 47 | 236 |
| 59 | −17 | 66 |
| 60 | 64 | 177 |
| 61 | 52 | 145 |
| 62 | 55 | 141 |
| 63 | 9 | 60 |
| 70 | 188 | 349 |
| 81 | 109 | 205 |
| 89 | 23 | 69 |
| 90 | 60 | 164 |
| 91 | 55 | 84 |
| 92 | 27 | 57 |
| 93 | 17 | 81 |
| 96 | 119 | 162 |
| 98 | 53 | 97 |
| 99 | 58 | 121 |
| 113 | 63 | 88 |
| 114 | 108 | 129 |
| 115 | 26 | 66 |

1. A compound of the formula I 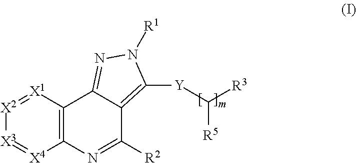
in which either
R³ represents an optionally substituted aryl group, an optionally substituted cycloalkyl group, an optionally substituted heteroaryl group, an optionally substituted heterocyclyl group; a substituted alkyl group and
m represents 0, 1, 2 or 3
or
R³ represents hydrogen and
m represents 2, 3, 4, 5 or 6;
and
R⁵ represents hydrogen or alkyl;
Y represents O or S;
R¹ represents an optionally substituted aryl group, an optionally substituted cycloalkyl group or an optionally substituted alkyl group;
X¹ represents N, CR⁴;
X² represents N, CR⁴;
X³ represents N, CR⁴;
X⁴ represents N, CR⁴;
R⁴ represents hydrogen or a substituent different from hydrogen;
R² represents hydrogen or a substituent different from hydrogen;
and
provided that not more than two of X¹-X⁴ represent nitrogen;
in free base form or in acid addition salt form.

2. A compound of formula I according to claim 1 wherein
R³ represents an aryl group or a (C₃-C₈)cycloalkyl group or a heterocyclyl group with 3 to 8 ring atoms or a heteroaryl group with 3 to 8 ring atoms or a (C₁-C₈)alkyl group;
wherein said aryl group, (C₃-C₈)cycloalkyl group, heteroaryl group, heterocyclyl group is unsubstituted, mono-substituted, di-substituted or tetra-substituted, the optional substituent(s) being independently selected from the group consisting of halogen, (C₁₋₈)alkyl, (C₁₋₈)alkyl substituted by halogen, (C₃₋₈)cycloalkyl, (C₃₋₈)cycloalkyl(C₁₋₈)alkyl, (C₁₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₄)alkylamino(C₁₋₈)alkyl with two identical or different (C₁₋₈)alkyl moieties in the di(C₁₋₈)alkylamino moiety, amino(C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, formyl, (C₁₋₈)alkylcarbonyl, formyloxy, (C₁₋₈)alkylcarbonyloxy, formyl(C₁₋₄)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl, (C₁₋₈)alkoxycarbonyl(C₁₋₈)alkoxy, —OCH₂O—, —C(═O)OCH₂—, —CH₂C(═O)— and —CH═CHCH═CH—, the four last-mentioned optional substituents in each case being attached to two adjacent ring carbon atoms of the said moiety and
wherein said (C₁₋₈)alkyl group is mono-substituted or di-substituted, the optional substituent(s) on the said (C₁₋₈)alkyl moiety being independently selected from the group consisting of halogen, cyano, oxo, (C₁₋₈)alkoxy, (C₁₋₈)alkoxy(C₁₋₈)-alkoxy, (C₁₋₈)alkylthio, (C₁₋₈)alkylsulfinyl, (C₁₋₈)alkylsulfonyl, (C₁₋₈)alkylcarbonyloxy, (C₁₋₈)alkoxycarbonyl and (C₁₋₈)alkoxy carbonyloxy and
R⁵ represents hydrogen or (C₁₋₄)alkyl and
m represents 0, 1 or 2 if R³ is a substituent as defined above other than hydrogen;
or
R³(CHR⁵)ₘ— represents ethyl, n-, iso-propyl, n-, iso-, sec.-, tert.-butyl, n-, sec.-neo.-, iso-pentyl, n-, iso-, sec.-hexyl
and
R¹ represents an optionally mono-, di-, tri- or tetra-substituted aryl group, an optionally mono-, di-, tri- or tetra-substituted cycloalkyl group or an optionally mono-, di-, tri- or tetra-substituted alkyl group; the substituent(s) being independently selected from the group consisting of halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino(C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, morpholino(C₁₋₈)alkoxy, piperidino(C₁₋₈)alkoxy, pyrrolidino(C₁₋₈)alkoxy, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl, (C₁₋₈)alkoxycarbonyl(C₁₋₈)alkoxy and —CH═CHCH═CH—, the last-mentioned optional substituent being attached to two adjacent ring carbon atoms of the said aryl group;
R² is selected from the group consisting of hydrogen, halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₄)alkyl, (C₁₋₈)alkylsulfonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino (C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl and (C₁₋₈)alkoxycarbonyl(C₁₋₈)alkoxy;
Each R⁴ is independently selected from the group consisting of hydrogen, halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino, (C₁₋₈)alkoxy, (C₁₋₈)alkylamino (C₁₋₈)alkoxy, di(C₁₋₈)alkylamino (C₁₋₈)alkoxy with two identical or different (C₁₋₈)alkyl moieties, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl and (C₁₋₈)alkoxycarbonyl(C₁₋₈)alkoxy or heteroaryl;
Y represents O.

3. A process for the preparation of a compound of the formula I as defined in claim 1, in free base form or in acid addition salt form, comprising the steps of
A) reacting of a compound of the formula II 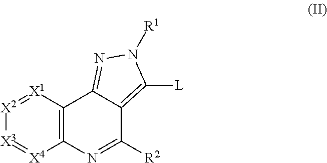
wherein the substituents are as defined for the formula I in claim 1 and L represents a leaving group, such as a halogen, tosylate, mesylate, with a compound of the formula III 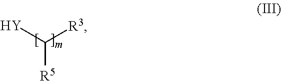
wherein the substituents are as defined for the formula I in claim 1, optionally in the presence of a base, such as a hydride; optionally in the presence of one or more diluents;
or
B) reacting of a compound of the formula IV 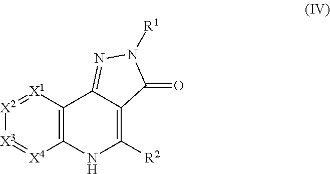
wherein the substituents are as defined for the formula I, with POCl₃
followed by a reaction with a compound of the formula III 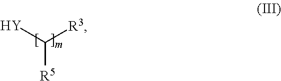
wherein the substituents are as defined for the formula I in claim 1, optionally in the presence of a base, such as a hydride; optionally in the presence of one or more diluents;
and
optionally followed by reduction, oxidation or functionalisation reaction of the resulting compound of formula I and/or by cleavage of protecting groups optionally present,
and
optionally followed by recovering the so obtainable compound of the formula I in free base form or in acid addition salt form.

4-6. (canceled)

7. A method for the treatment, prevention or delay of progression of a condition, disease or disorder, that can be modulated or is mediated by GABA-A receptors, comprising administering to a subject in need thereof a therapeutically effective amount of a compound of the formula I as defined in claim 1, in free form or in pharmaceutically acceptable salt form.

8. A pharmaceutical composition comprising a compound of the formula I as defined in claim 1, in free form or in pharmaceutically acceptable salt form, as active ingredient, and a pharmaceutical carrier or diluent.

9. A pharmaceutical composition comprising a therapeutically effective amount of a compound of the formula I as defined in claim 1, in free form or in pharmaceutically acceptable salt form, and a second drug substance.
